(19) United States
(12) Patent Application Publication
(10) Pub. No.: US 2018/0125950 A1
(43) Pub. Date: May 10, 2018

(54) LIVER CIRRHOSIS TREATMENT

(71) Applicants: Mubashir H. Khan, Springfield, MO (US); Jesse G. Taylor, Nixa, MO (US)

(72) Inventors: Mubashir H. Khan, Springfield, MO (US); Jesse G. Taylor, Nixa, MO (US)

(21) Appl. No.: 15/810,250

(22) Filed: Nov. 13, 2017

Related U.S. Application Data
(63) Continuation-in-part of application No. 15/639,250, filed on Jun. 30, 2017.
(60) Provisional application No. 62/343,280, filed on May 31, 2016.

(51) Int. Cl. A61K 38/48; A61M 5/32; A61B 17/3207; A61K 9/00

(52) U.S. Cl. CPC A61K 38/4886; A61M 5/329; A61B 17/320725; A61B 2017/00818; A61K 9/0019; A61M 5/3286; C12Y 304/24

(57) ABSTRACT

A method of treating liver cirrhosis has ‘micro-cuts’ being created within the parenchyma of a cirrhotic liver to create ‘micro-cavities’, so that hepatocytes have more ‘room’ to regenerate; and thus an overall increase in hepatocyte volume occurs. Alternatively, the method of treating liver cirrhosis has fibrotic collagen being disrupted enzymatically (eg collagenase) through localized intra-parenchymal injections. The increase in hepatocyte volume at focal sites of mechanical fibrosis disruption (via ‘cuts’ or ‘collagenase’) provides for a ‘whole-organ’ treatment of a cirrhotic liver.

### CROSS-REFERENCE TO PROVISIONAL APPLICATION(S)

This application is a continuation-in-part of U.S. patent application Ser. No. 15/639,250, filed Jun. 30, 2017; which claims the benefit of U.S. Provisional Application No. 62/343,280, filed May 31, 2016. The foregoing patent disclosures are incorporated herein by this reference thereto.

### BACKGROUND AND SUMMARY OF THE INVENTION

The invention relates to surgery and, more particularly, to a method of treating liver cirrhosis.

Whereas research has been performed evaluating various biochemical and cellular aspects of hepatocyte regeneration and fibrosis in liver cirrhosis, data evaluating mechanical disruption of fibrotic tissue in a cirrhotic liver and resultant effects on hepatocyte regeneration is sparse or unavailable. Reversal or ‘cure’ of cirrhosis remains challenging in the clinical setting and currently, liver transplantation remains the mainstay of treatment options available to recover hepatic function in end-stage liver disease.

It is an aspect of the invention to preform localized/focal mechanical disruption of hepatic fibrosis within regions of the liver in order to lead to viable hepatocyte regeneration by relieving constricting pressures on hepatocyte clusters.

It is another aspect of the invention to enzymatically disrupt (eg., collagenase) fibrotic collagen through localized intra-parenchymal injections thereby increasing hepatocyte.

The increase in hepatocyte volume at focal sites of mechanical fibrosis disruption (via ‘cuts’ or ‘collagenase’) provides for a ‘whole-organ’ treatment of a cirrhotic liver.

A number of additional features and objects will be apparent in connection with the following discussion of the preferred embodiments and examples with reference to the drawings.

### BRIEF DESCRIPTION OF THE DRAWINGS

There are shown in the drawings certain exemplary embodiments of the invention as presently preferred. It should be understood that the invention is not limited to the embodiments disclosed as examples, and is capable of variation within the scope of the skills of a person having ordinary skill in the art to which the invention pertains. In the drawings,

FIG. 1 is an illustration—partly in perspective on a relatively reduced scale, in contrast to—partly diagrammatic on a relatively enlarged scale—of apparatus in accordance with the invention for performing a method in accordance with the invention for treating liver cirrhosis, and, showing (in the upper half of the view) an injector/injection needle which also has provisions to function as a “core creator,” for creating (in the lower half of the view) ‘micro-cuts’ within the parenchyma of a cirrhotic liver to create ‘micro-cavities’, so that hepatocytes have more ‘room’ to regenerate, wherein fibrotic collagen is disrupted enzymatically (eg., collagenase) through localized intra-parenchymal injections;

FIG. 2 is an enlarged scale perspective view of the injector/injection needle also serving (in combination) as a “core creator” as shown FIG. 1, wherein the needle has a narrow gauge flat beveled (or otherwise beveled) injection tip;

FIG. 3 is a perspective view comparable to FIG. 2 except showing that the needle/“core creator” in accordance with the invention has folded-in blades which can be folded-out as shown, with blades being deployed out at instances when used for providing “core” cuts in liver tissue;

FIG. 4 is a perspective view comparable to FIG. 2 except exploded;

FIG. 5 is an enlarged scale section view taken along line V-V in FIG. 2, showing that the needle shank has a triple sleeve construction, wherein the innermost sleeve ultimately terminates in the beveled tip, and the innermost and outermost sleeves are fixed relative to each other, but that the intermediate sleeve can be translated back forth inside an annular gap spacing apart the innermost and outermost sleeves, ie/, as in back and forth between extension and retraction strokes;

FIG. 6 is an enlarged scale section view taken along line VI-VI in FIG. 5;

FIG. 7 is an enlarged scale section view of detail VII-VII in FIG. 5, and showing the rack-and-pinion drive configuration for deploying a representative blade, wherein the blade is pivoted at its base end by a pivot pin in the outermost sleeve, which base end has pinion gear teeth, and wherein the intermediate sleeve is formed with rack teeth to engage the pinion gear teeth of the base of the blade, and hence drive the blade between folded-out and folded-in extremes;

FIG. 8 is an enlarged scale section view taken along the line VIII-VIII in FIG. 7;

FIG. 9 is an enlarged scale perspective view, partly in section, taken from detail IX-IX in FIG. 3, showing the transition portion of the injector/“core creator” from the syringe reservoir portion thereof to the elongated, triple-sleeve needle shank thereof;

FIG. 10 is an enlarged scale perspective view, partly in section, taken in the direction of arrows X-X in FIG. 2, showing a central drive shaft for the syringe and an annular drive shaft for the intermediate sleeve;

FIG. 11 is a section view comparable to FIG. 5 except showing an alternate embodiment of the subject matter previously shown in FIG. 7, which alternate embodiment is more particularly shown next in FIG. 12;

FIG. 12 is an enlarged scale section view taken from the detail XII-XII in FIG. 11 and showing an alternate embodiment of the subject matter previously shown in FIG. 7, wherein deployment of the representative blade hereof is caused by a drive slot in the intermediate pin driving/cranking a crank pin on the pivoted base end of the blade;

FIG. 13 is an enlarged scale perspective view of FIG. 12 and partly broken away to show the triple sleeve construction of needle shank in accordance with the invention;

FIG. 14 is a perspective view comparable to FIG. 13, except partly in section;

FIG. 15 is an enlarged scale perspective view, partly in section, of detail XV-XV in FIG. 14;

FIG. 16 is a perspective view comparable to FIG. 13 except partly exploded, wherein, this FIG. 16 shows the scalpel-edge sharpness of the blades;

FIG. 17 is a reduced scale section view comparable to FIGS. 5 and 11 except showing an alternate embodiment of the subject matter previously shown in FIGS. 7 and 12, which alternate embodiment is more particularly shown next in FIG. 18;

FIG. 18 is an enlarged scale section view taken from the detail XVIII-XVIII in FIG. 17 and showing an alternate embodiment of the subject matter previously shown in FIGS. 7 and 12;

FIG. 19 is a perspective view, partly in section, comparable to FIG. 14, except showing the FIGS. 17 and 18 embodiment, wherein the blades here comprise flexible tangs formed in the intermediate sleeve and deploy through ramped exit openings in the outermost sleeve;

FIG. 20 is a perspective view, partly in section, comparable to FIG. 19, except showing the triple-sleeve construction of the needle in a partial stage of deployment whereby the folded-in blade is in a relatively extreme folded-in state;

FIG. 21 is a perspective view, partly in section, comparable to FIGS. 19 and 20, except showing the triple-sleeve construction of the needle shank in a further of deployment whereby the flexible blade tang is partially folded-out in a stage of folding out;

FIG. 22 is an enlarged scale section view taken along line XXII-XXII in FIG. 20;

FIG. 23 is a section view comparable to FIG. 22 except showing a variation with a wider blade tang;

FIG. 24 is an enlarged scale perspective view comparable to FIG. 3 except with the syringe portions broken away, and in tandem with an end view, showing the multiplicity of blades to be arranged in a first and second single-file row of a plurality of blades angularly-spaced 180° apart on the triple-sleeve construction of the needle, with each blade of one row having an opposite counterpart in a ring with the other row;

FIG. 25 is a tandem perspective view and end view comparable to FIG. 24 except showing the multiplicity of blades arranged in a three single-file rows of a plurality of blades angularly-spaced 120° apart on the triple-sleeve construction of the needle shank, with each blade of one row having two respective counterparts in a ring with the respective other rows;

FIG. 26 is an end view comparable to the end view of FIG. 24 except showing the multiplicity of blades arranged in a eight single-file rows of a plurality of blades angularly-spaced 45° apart on the triple-sleeve construction of the needle shank, with each blade of any of the 12 o'clock, 3 o'clock, 6 o'clock and 9 o'clock rows having three respective counterparts in a ring the other of those respective rows, which are axially-spaced apart and axially alternate with four counterpart blades in the 1:30, 4:30, 7:30 and 10:30 rows, which form their own rings;

FIG. 27 is a tandem perspective view and end view comparable to FIG. 24 except showing the multiplicity of blades arranged in file along two helical coiling rows of a plurality of blades, which helically coiling rows are angularly-spaced 180° apart at any axial station on the on the triple-sleeve construction of the needle shank, with each blade of one helical coil having an opposite counterpart in the other helical coil forming their own ring;

FIG. 28 is a tandem perspective view and end view comparable to FIG. 24, and showing the multiplicity of blades being arranged in a first and second single-file row of a plurality of blades angularly-spaced 180° apart on the triple-sleeve construction of the needle shank, with each blade of one row having an opposite counterpart in a ring with the other row, except wherein the axial spacing of the blades and radial-extreme extensions for the blades much more abbreviated and concentrated toward the beveled tip end of the needle;

FIG. 29 is a tandem perspective view and end view comparable to FIG. 28, except showing a plurality of blades at angularly-spaced positions 90° apart, and there being only one ring of blades, hence four blades overall; concentrated very close to the beveled tip end of the needle;

FIG. 30A is a diagrammatic view of a method in accordance with the invention for treating liver cirrhosis, and showing ‘micro-cuts’ being created within the parenchyma of a cirrhotic liver to create ‘micro-cavities’, so that hepatocytes have more ‘room’ to regenerate;

FIG. 30B is a diagrammatic view of a variation of the method in accordance with the invention for treating liver cirrhosis, and showing fibrotic collagen being disrupted enzymatically (eg collagenase) through localized intra-parenchymal injections;

FIG. 31 is an enlarged scale diagrammatic view showing a resultant “core” cut in liver tissue; and

FIG. 32 is a diagrammatic view of a liver scored with a plurality of “cores” formed by the method in accordance with the invention, over several treatment sessions.

### DETAILED DESCRIPTION OF THE PREFERRED EMBODIMENTS

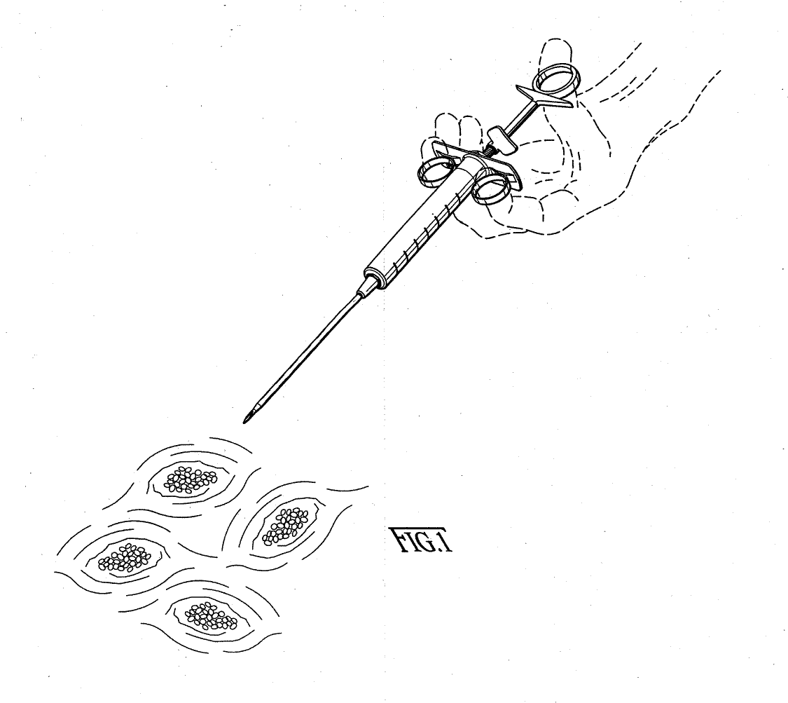

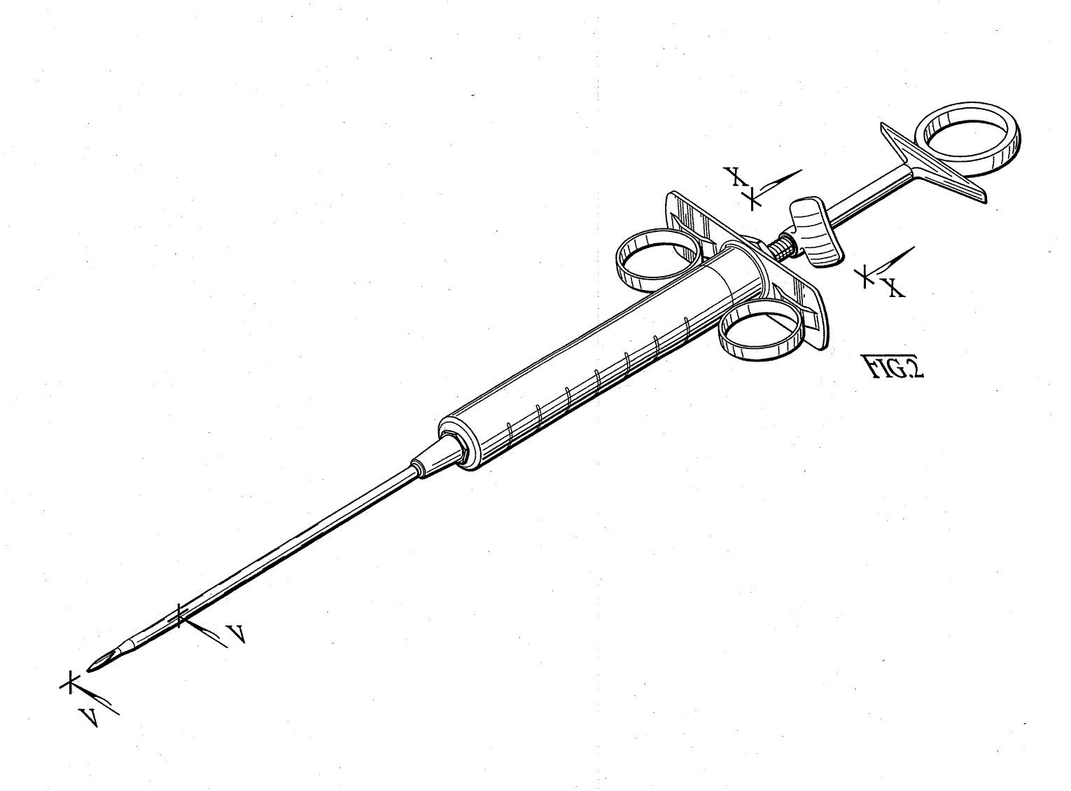

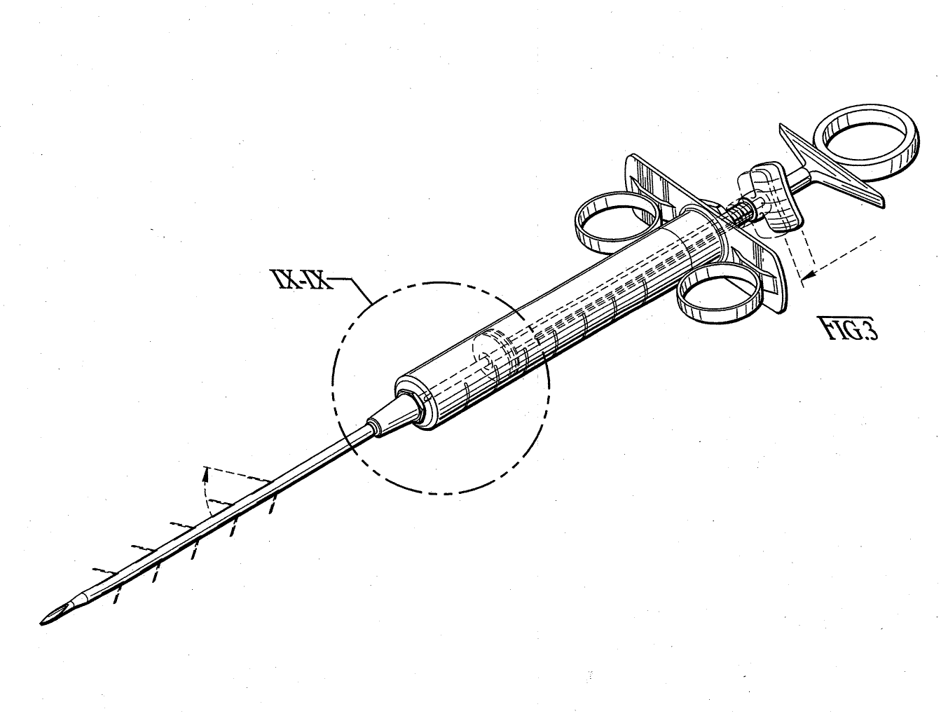

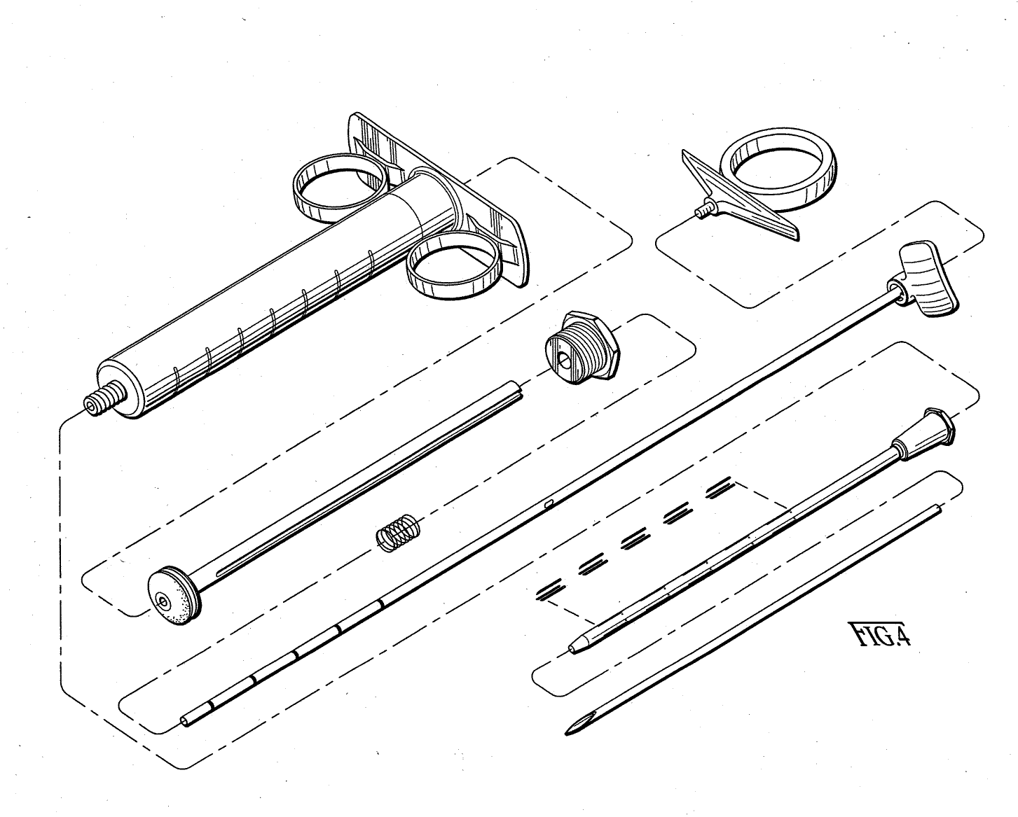

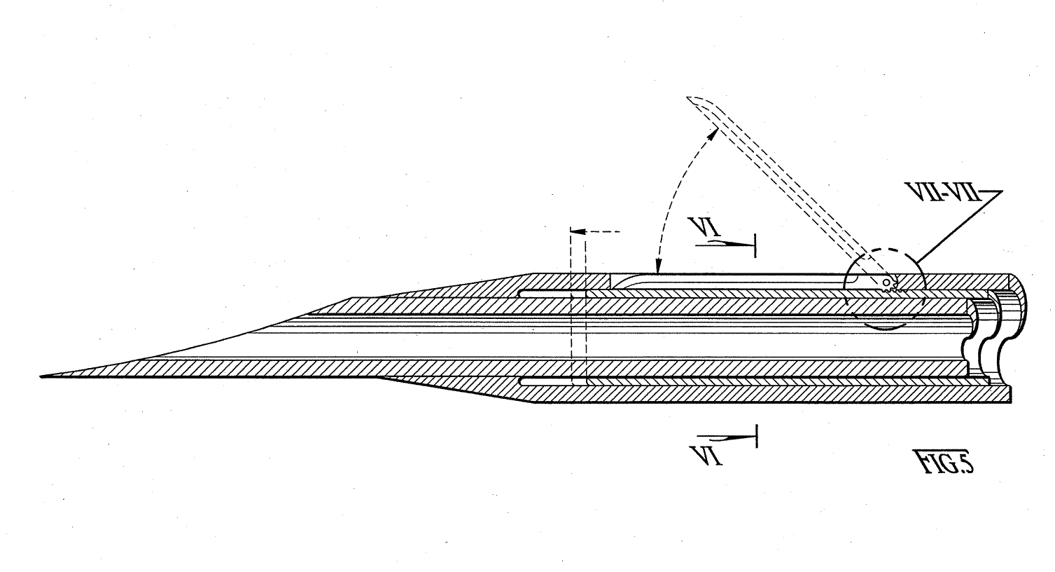

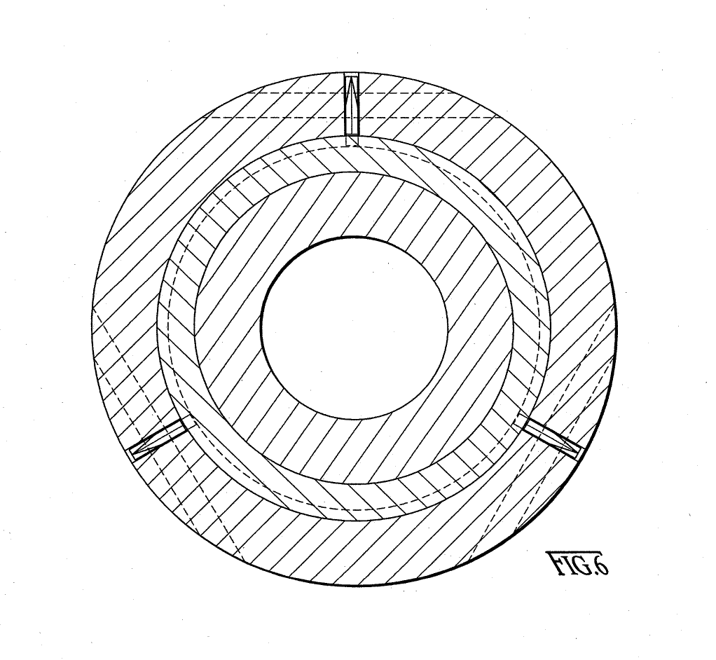

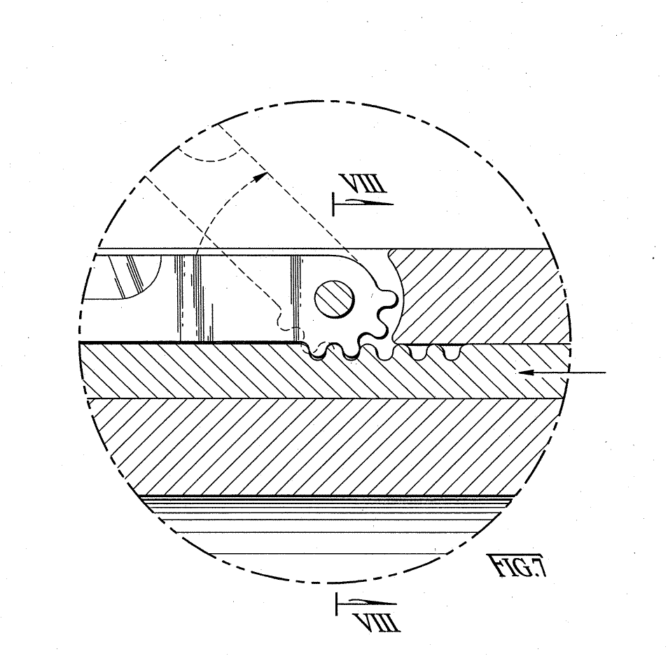

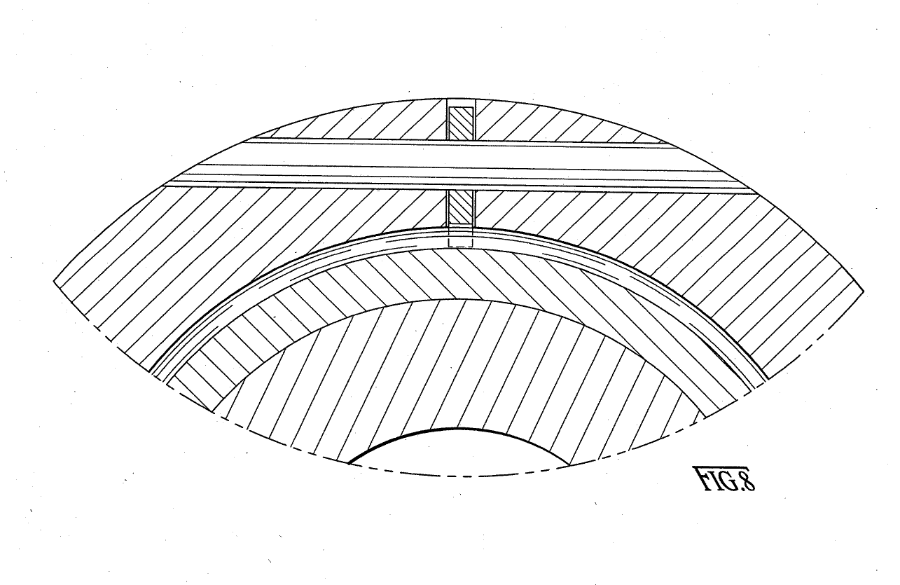

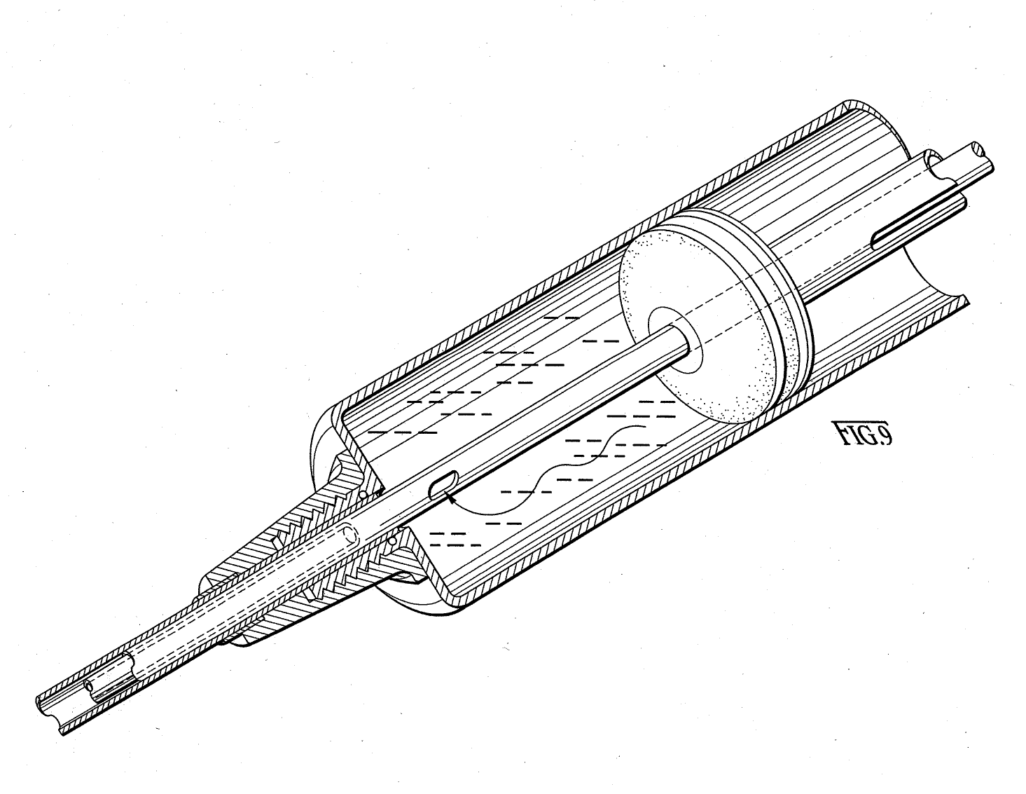

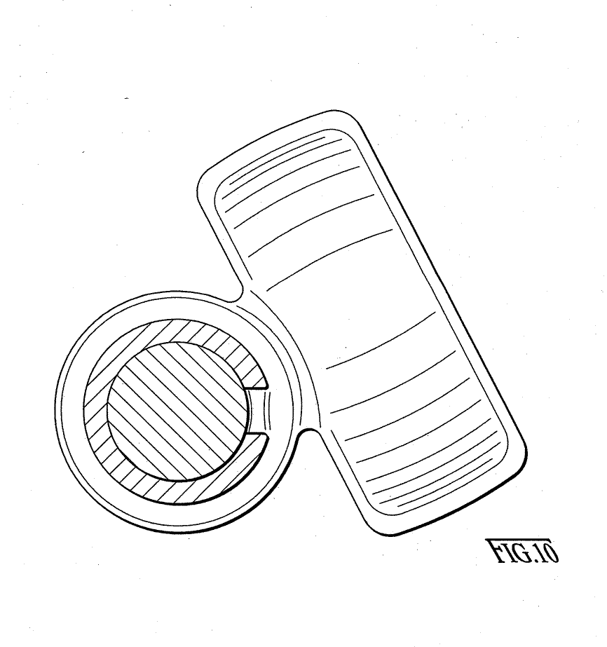

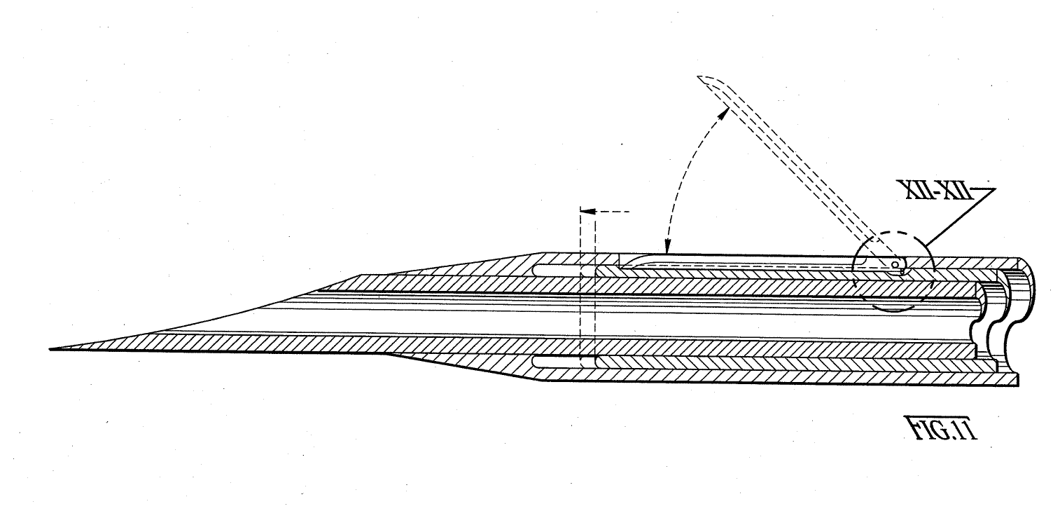

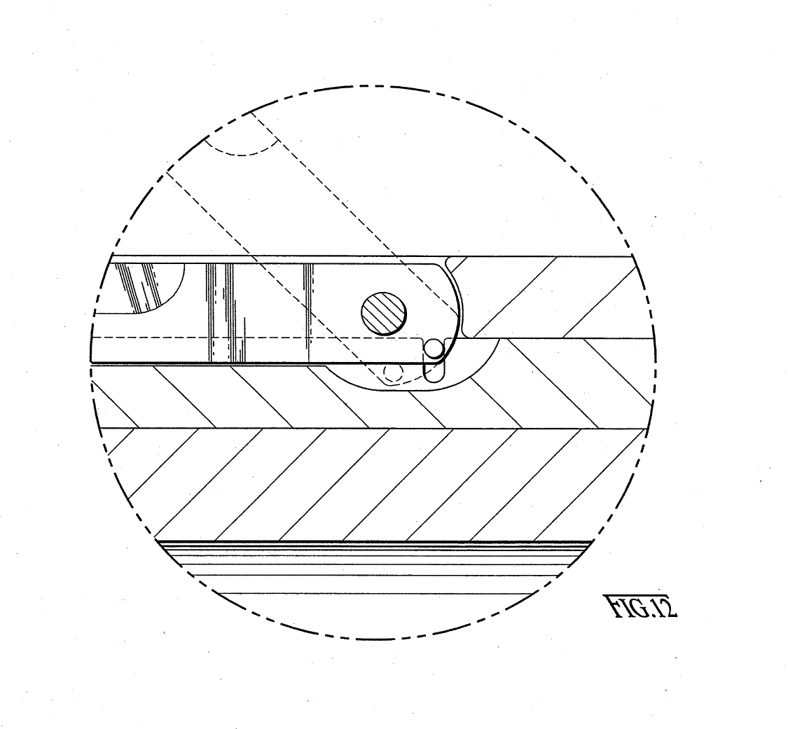

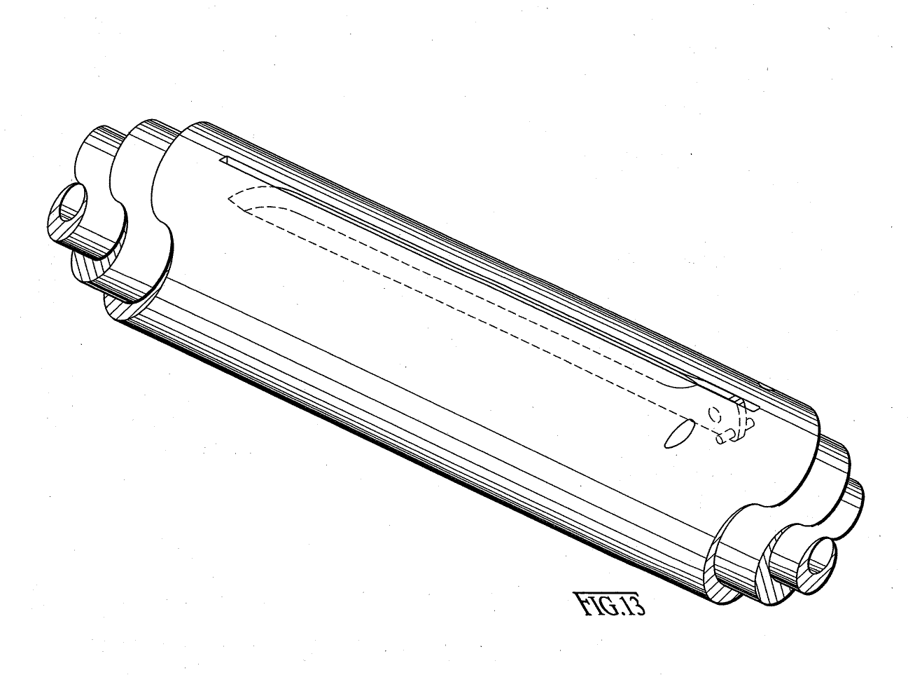

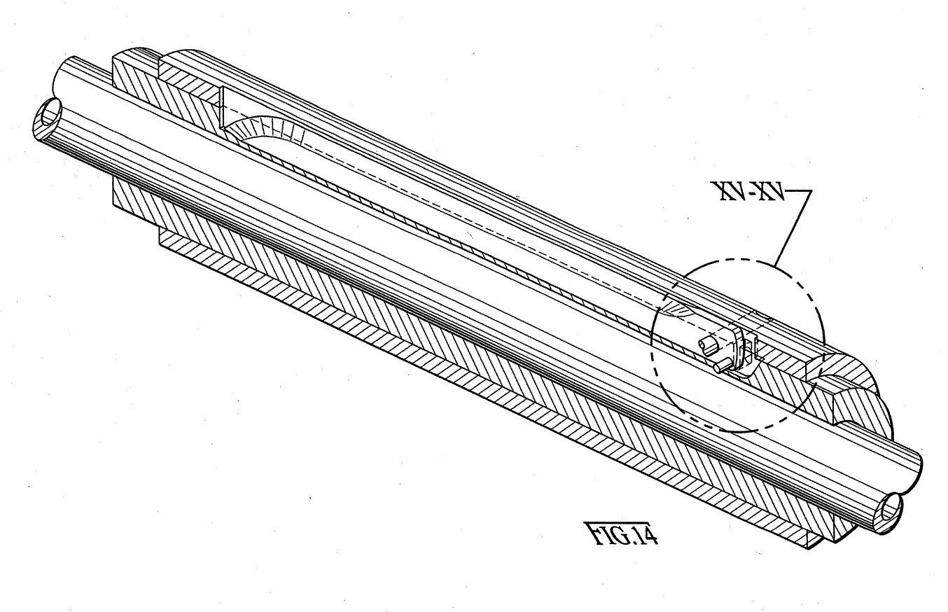

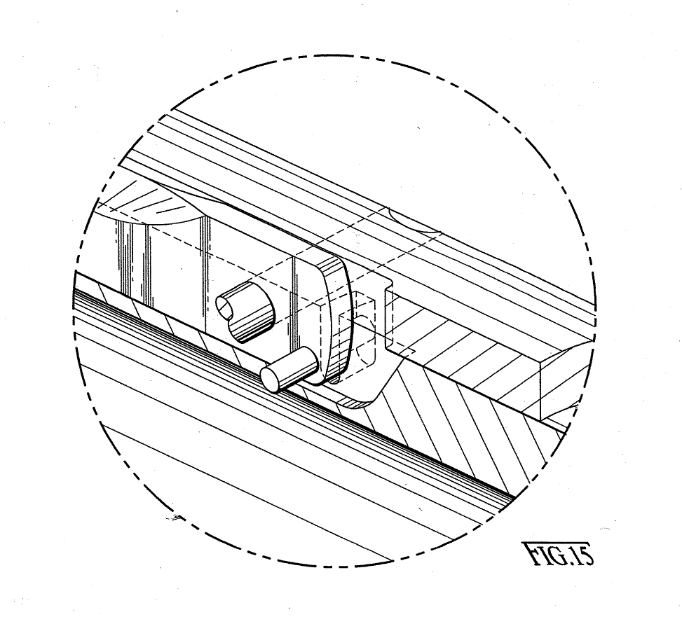

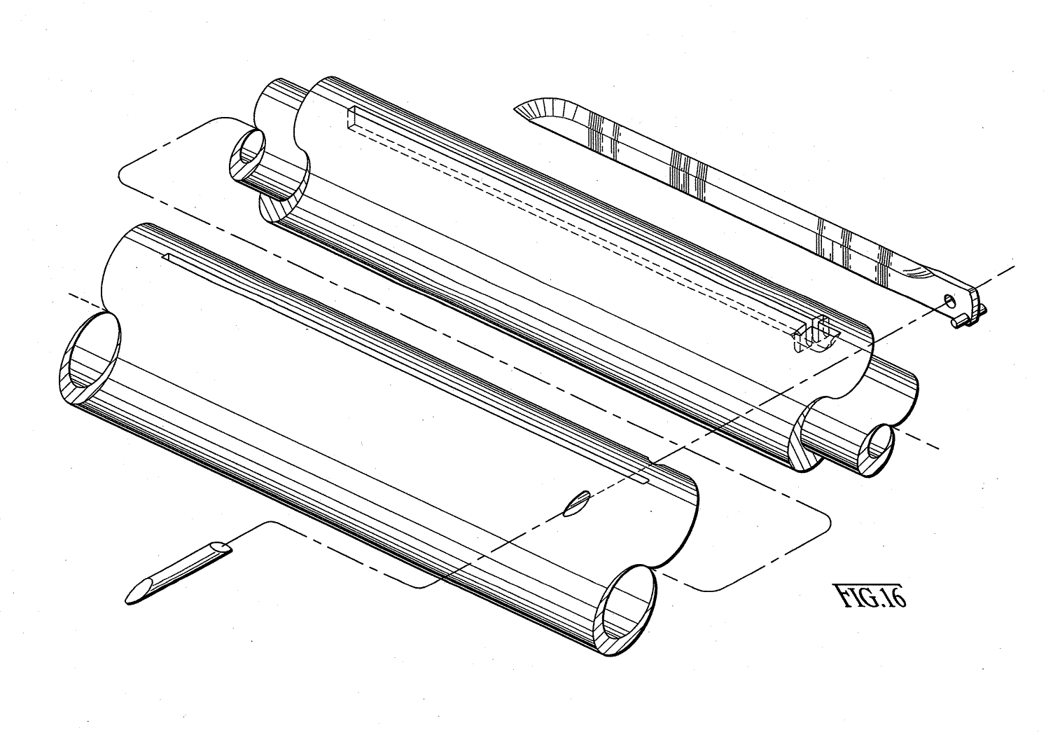

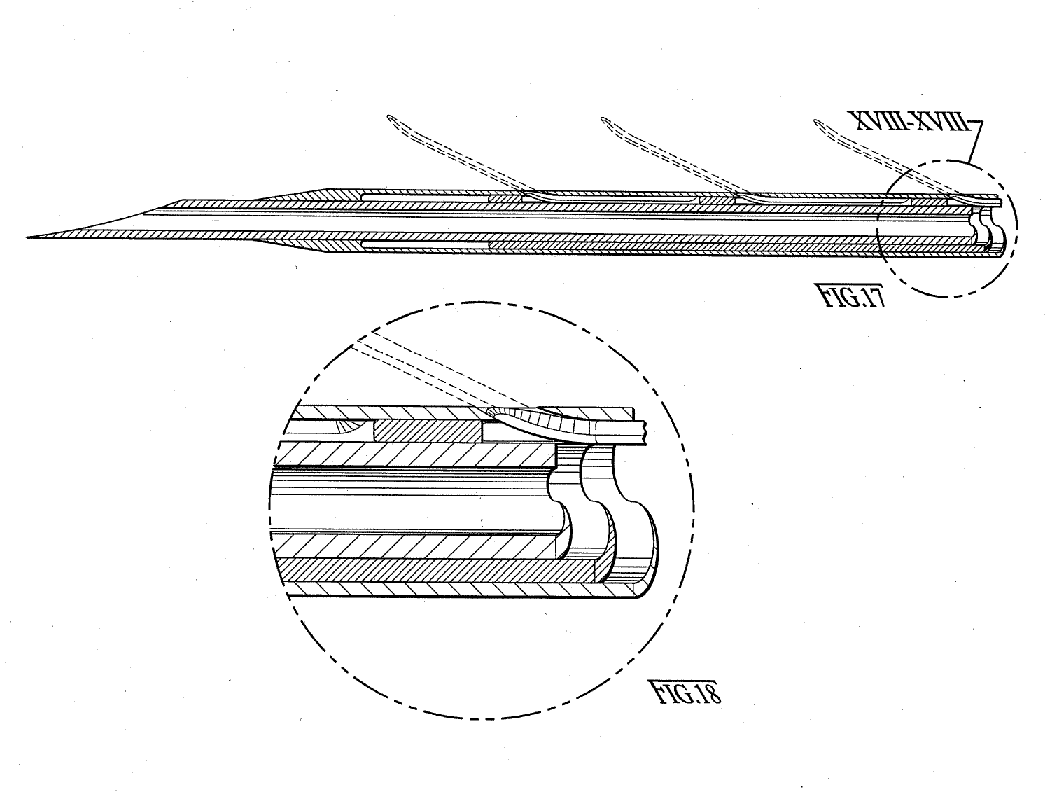

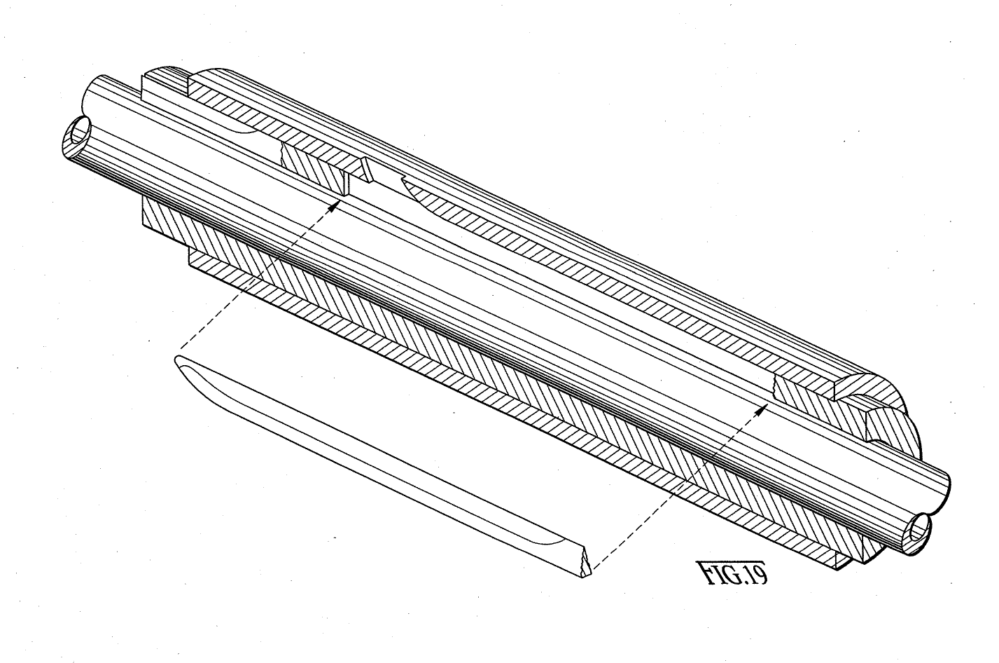

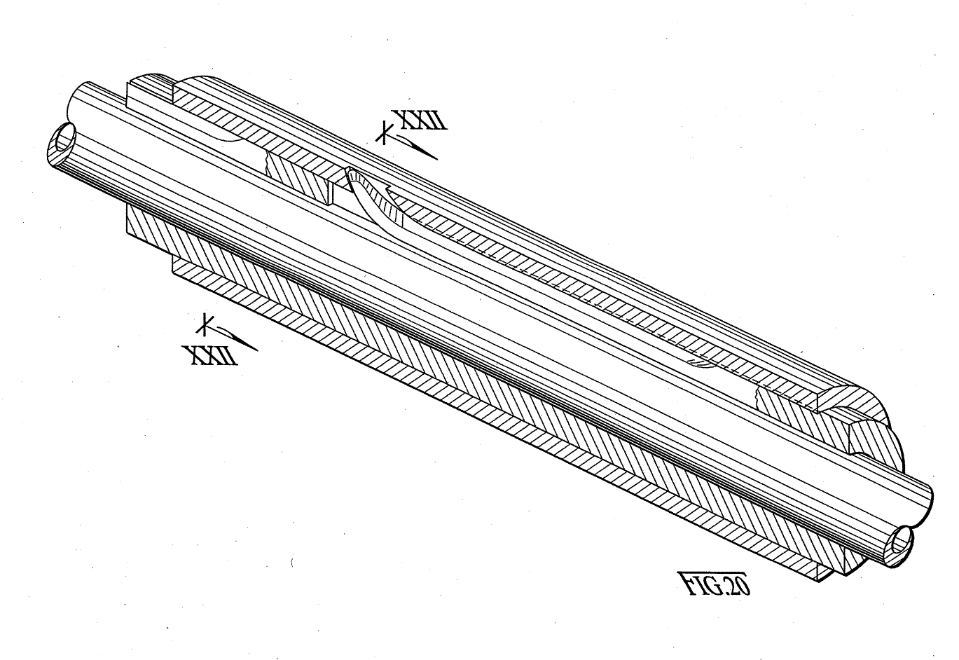

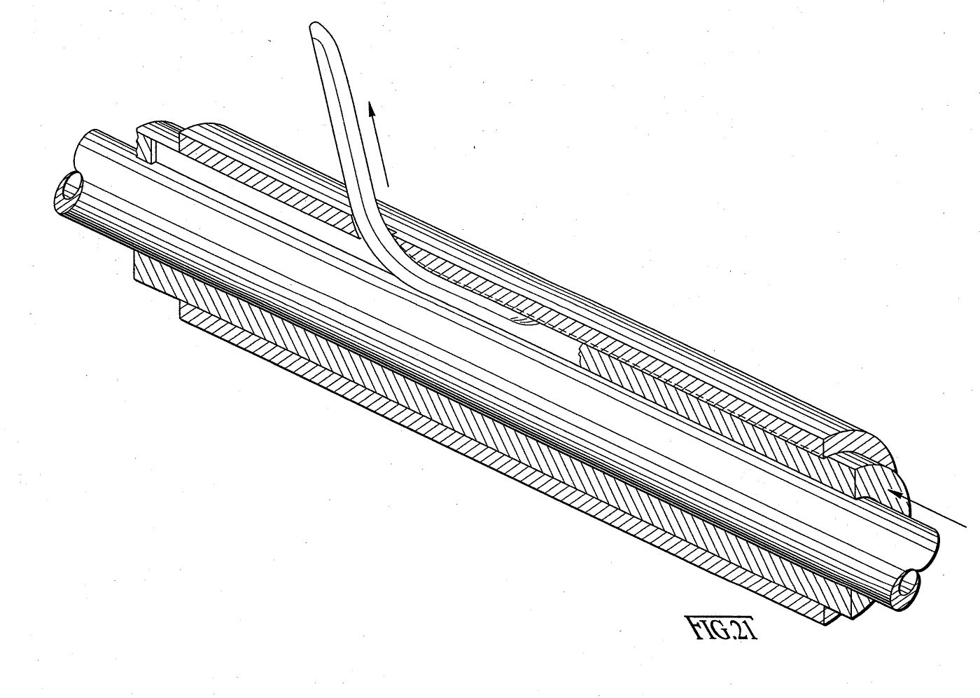

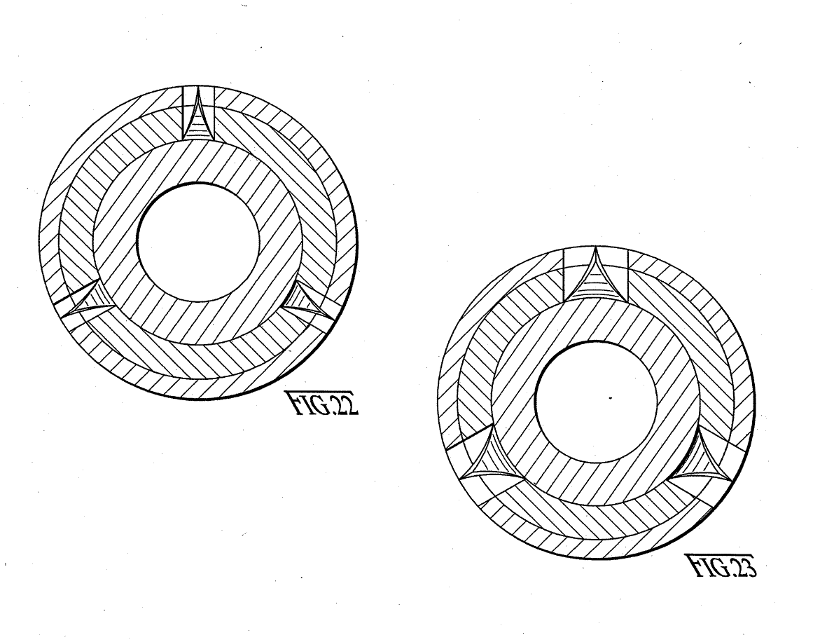

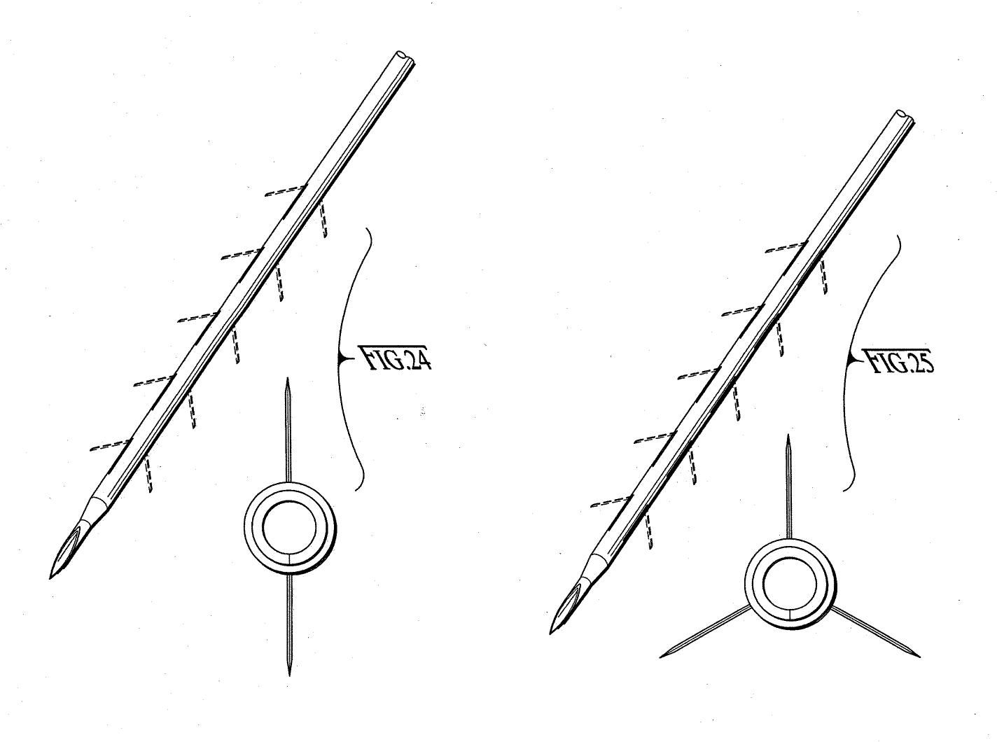

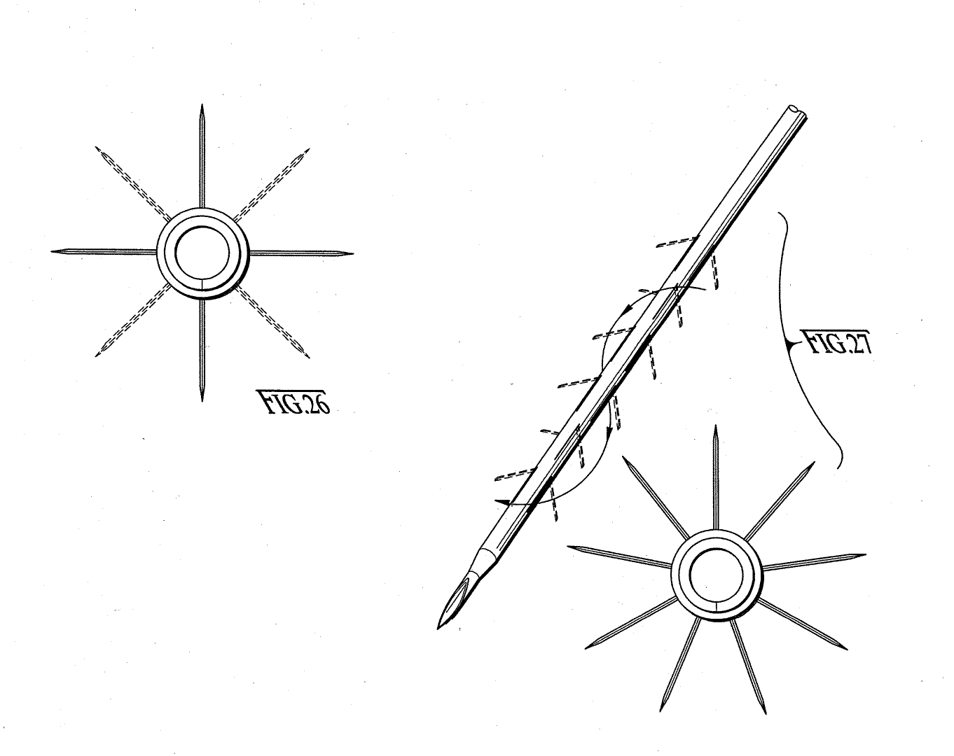

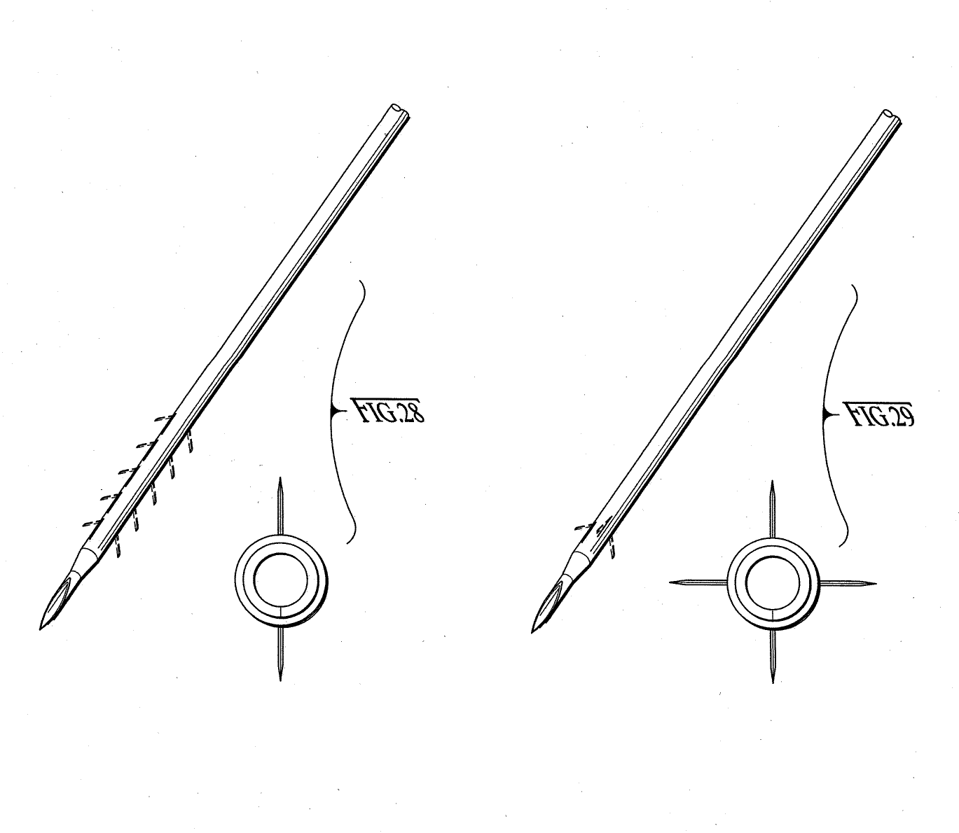

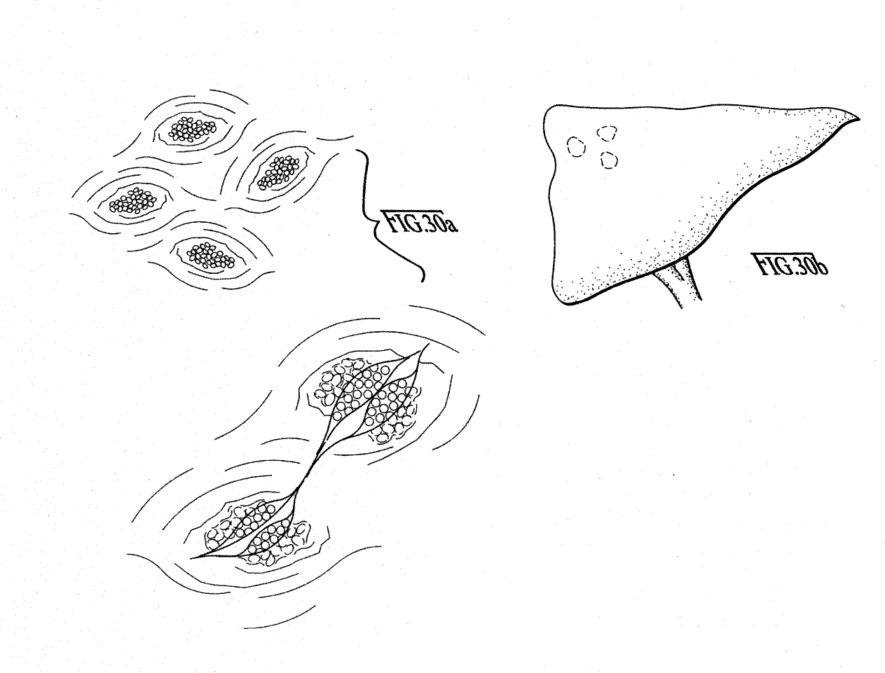

With reference to FIG. 1, it is hypothesized that localized/focal mechanical disruption of hepatic fibrosis within regions of the liver may lead to viable hepatocyte regeneration by relieving constricting pressures on hepatocyte clusters. If ‘micro-cuts’ could be created within the parenchyma of a cirrhotic liver to create ‘micro-cavities’, hepatocytes may have more ‘room’ to regenerate; and thus an overall increase in hepatocyte volume may occur. Alternatively, fibrotic collagen can be disrupted enzymatically (eg collagenase) through localized intra-parenchymal injections. If increase in hepatocyte volume at focal sites of mechanical fibrosis disruption (via ‘cuts’ or ‘collagenase’) can be demonstrated through lab research, new therapies for ‘whole-organ’ treatment of a cirrhotic liver could subsequently be designed. (See FIGS. 30A/30B.)

Many complex, resource-intensive experiments (involving teams of hepatologists, radiologists, pathologists etc) can be designed to study this phenomenon further. A cirrhosis animal model would be needed. Then, exact ‘micro-cuts’ or localized collegen injections within image-mapped liver fields away from blood vessels and large ducts could be performed. Subsequently, these exact image-mapped areas could be studied to evaluate for evidence of hepatocyte regeneration (at sites of fibrosis disruption). These detailed experiments may require large grants and infrastructure to conduct.

However, ‘quick-and-dirty’ initial experiments to study this issue could be designed cheaply as follows. A laboratory rat cirrhosis model (CCl4) could be used. Simple accessory requirements would include:—
a) to create mechanical rupture of intra-parenchymal fibrotic tissue: sterile narrowest gauge flat beveled injection needles (these have blade-like tips (see FIG. 5) for ‘micro-cutting’ the liver parenchyma; or, collagenase. If needles are used, the tip (cutting edge) diameter should be at least 2-4 times that of an average liver micro nodule (so that complete disruption of fibrosis surrounding micro clusters could occur);
b) to identify location of ‘micro-cut’ or collagenase action post-harvest for histology: micro-pipettes containing medical grade dye for ‘internal marking’ of the micro-cut tracts of the liver, or, the sites of collegenase injections;
c) possibly Hepatocyte-Growth Factor; or other hepatocyte growth factors; and
d) liver harvesting and pathology slide capabilities.

### Experiment Design: ‘Cuts’ or ‘Collagenase’ Options.

e) Though not necessary, cirrhotic rats could be primed for a period of time with HGF prior to undertaking manipulation of liver parenchyma. External landmarks on experimental cirrhotic animals are reviewed to determine the location of the liver. The narrow-gauge needle is inserted percutaneously blindly into the cirrhotic liver for a range of for e.g. 3-5 mms depending on animal (also multiple animals will be used; e.g. 10 rats). This process would exactly be analogous to when a percutaneous liver biopsy is performed using external landmarks. On the way in, the needle should automatically ‘micro-cut’ through a track of fibrous bands in the liver. A nano-milliliter of dye is injected before the needle is withdrawn to identify the location of intervention. In another variation, ‘micro-globule(s)’ of collagenase could be injected within the cirrhotic liver parenchyma. Each injected ‘globule’ would occupy a space of a few millimeters and should theoretically weaken/destroy the collagen within its area of action. These areas can also be tattooed with dye mixed with collagenase at time of initial injection.
f) If either of the aforementioned variations of these procedures are performed in lets say, 10 animals; there is bound to be significant early mortality due to the ‘blind’ technique of initial needle insertion inevitably leading to blood-vessel injury, duct damage, or, other complications. However, a significant fraction of animals would survive. The livers of the surviving animals will be harvested in a ‘staggered’ manner at 2-4 week post-intervention (′ cut′ or collagenase) interval. The tattooed track (or collagenase field) could be identified visually and this area of interest examined histologically to determine if and when new hepatocyte regeneration has occurred.
g) These ‘quick and dirty’ variations of experiments could be undertaken at little more than the cost of the cirrhosis-model animals used.
h) Similarly, other experiments which could be run to evaluate this same concept would include evaluating the role of collagenase injections into the portal vein for global hepatic effect; using small needle-tipped balloon dilators to percutaneously create ‘cavities’ to allow for hepatic regeneration, with or without injecting these ‘cavities’ with hepatic growth factors or even stem-cells. Other potential options would include deployment of implanting expandable metallic stent-like meshed devices within the liver parenchyma to create spaces promoting new ‘tissue-overgrowth’ within the cavities of these hollow devices. The latter are more exotic variations of this idea.
i) Preliminary histological data could potentially also be obtained from explant/autopsy specimens from deceased patients with cirrhosis who had undergone a TIPS or a liver biopsy (or other manipulation) 1-4 weeks prior to their death.

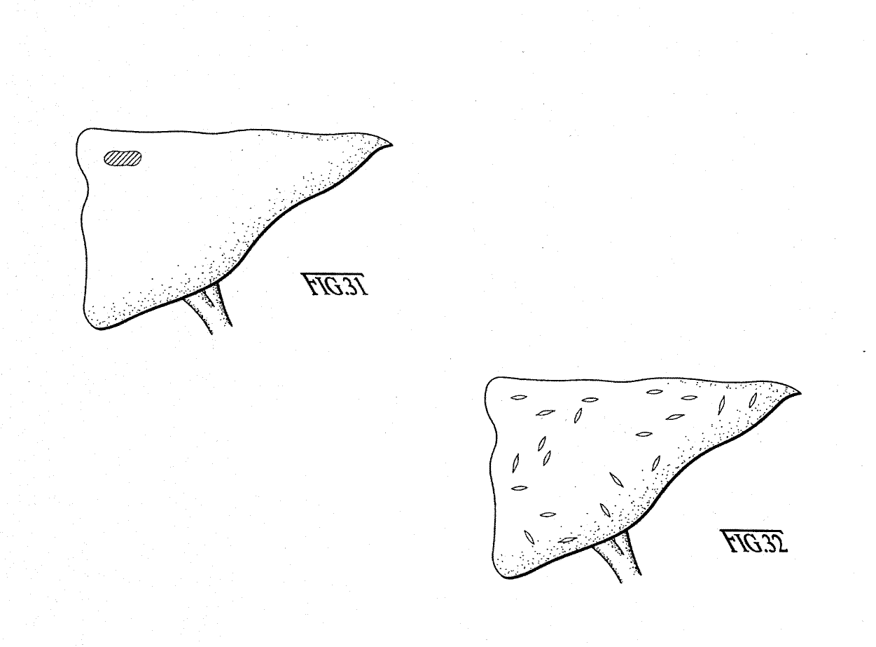

If initial experiments demonstrate that mechanical disruption of hepatic fibrous bands does in fact promote or lead to hepatocyte regeneration, further detailed experiments could be set up using image guided ‘treatments’ (via ‘cuts’ or collagenase) and larger animal models. As the scope expands further, the need to be able to treat multiple foci of fibrotic tissue within the same area of the liver efficiently and simultaneously would arise. To accomplish this may require invention of devices or specialized injection needles (for eg, see FIG. 31). Devices could be designed to be able to access the liver percutaneously, via the trans-jugular rout, or, via ERCP (to capture the maximum field of the fibrotic parenchyma). As an elaboration of one such concept, generation of ‘cores’ (see FIG. 32) of healthy liver tissue could be encouraged or accomplished (for e.g. using a variation if device in FIG. 31). Effect of ‘whole-organ’ treatments could then be studied for improvement in hepatic function. ‘Whole organ’ therapy would likely require multiple treatment ‘sessions’ so that acute hepatic decompensation is not triggered. By this stage in these series of experiments, significant funding or infrastructure would have been utilized. In the long run, more and more effective devices and technologies would be needed to make this treatment safe and effective. It is within the realm of possibility that patients in the future arrive for outpatient therapy ‘sessions’ using ‘cyber-knife’-like technologies after a diagnosis of liver cirrhosis is made; to see their hepatic functions improve over time as their liver expands in volume with ‘new’ hepatocyte cores being created via fibrosis disruption. Or, it may be a simple matter or injecting the right amount of collagen within the right locations within the liver to accomplish the same results.

An optimal outcome at the end of these series of experiments would entail gaining knowledge enabling one to mechanically enforce an increase in hepatic volume over time. This is hypothesized to lead to hepatic function recovery and resolution of portal hypertension.

Snake digestive physiology (eg Burmese python etc; also see for e.g. Sector SM. Jr Exp Biology 211 3767-3774;) offers interesting opportunities for the creation of a cirrhosis model for the study of hepatocyte regeneration and cirrhosis. The macroscopic hepatic volume response to a meals, when studied in such a model may offer useful insights into the disease and regeneration processes.

The invention having been disclosed in connection with the foregoing variations and examples, additional variations will now be apparent to persons skilled in the art. The invention is not intended to be limited to the variations specifically mentioned, and accordingly reference should be made to the appended claims rather than the foregoing discussion of preferred examples, to assess the scope of the invention in which exclusive rights are claimed.

We claim:

1. A method of treating liver cirrhosis comprises the steps of:
creating ‘micro-cuts’ within the parenchyma of a cirrhotic liver thereby creating ‘micro-cavities;’ and
affording hepatocytes more ‘room’ to regenerate, and thereby an overall increase in hepatocyte volume occurs.

2. A method of treating liver cirrhosis comprises the steps of:
disrupting fibrotic collagen enzymatically (eg., collagenase) through localized intra-parenchymal injections; and
inducing an increase in hepatocyte volume at focal sites of mechanical fibrosis disruption (via ‘cuts’ or ‘collagenase’) and thereby provide for a ‘whole-organ’ treatment of a cirrhotic liver.
